(19) United States
(12) Patent Application Publication
(10) Pub. No.: US 2024/0360065 A1
(43) Pub. Date: Oct. 31, 2024

(54) RECYCLED CONTENT DIOCTYL TEREPHTHALATE

(71) Applicant: Eastman Chemical Company, Kingsport, TN (US)

(72) Inventors: Kenneth James Flint, Roanoke, VA (US); Michael Neil Brown, Kingsport, TN (US); Hannah Grace Moser, Gate City, VA (US)

(73) Assignee: Eastman Chemical Company, Kingsport, TN (US)

(21) Appl. No.: 18/558,447

(22) PCT Filed: Apr. 21, 2022
(86) PCT No.: PCT/US2022/025660

§ 371 Date: Nov. 1, 2023

Related U.S. Application Data
(60) Provisional application No. 63/201,579, filed on May 5, 2021.

(51) Int. Cl. C07C 67/08

(52) U.S. Cl. CPC C07C 67/08

(57) ABSTRACT

Recycled content dioctyl terephthalate (r-DOTP) is produced using a process and system that applies credit-based recycled content from one or more feed materials to DOTP produced from the feed materials.

### BACKGROUND

Dioctyl terephthalate (DOTP) is a phthalate-free plasticizer that is considered safer than ortho-phthalate plasticizers due to its excellent toxicological profile. DOTP is often used for softening PVC plastics. Its primary uses are in applications like extrusion, calendaring, injection molding, rotational molding, dip molding, slush molding and coating.

The demand for recycled chemical products continues to grow, but there is no clear path to recycled DOTP through mechanical recycling. Thus, there exists a need for a commercial process to produce recycled DOTP.

### SUMMARY

In one aspect, the present technology concerns a process for producing dioctyl terephthalate (DOTP) having recycled content, where the process comprises the following steps: (a) reacting a propylene with a syngas to thereby provide an n-butyraldehyde; (b) converting at least a portion of the n-butyraldehyde into a 2-ethylhexanol; (c) reacting at least a portion of the 2-ethylhexanol with a terephthalyl to thereby provide a DOTP; and (d) applying recycled content to at least a portion of the DOTP to thereby provide a recycled content DOTP (r-DOTP). The applying of step (d) includes (i) attributing recycled content from at least one source material having physical recycled content to at least one target material via recycled content credits, (ii) tracing recycled content along at least one chemical pathway from the at least one target material to the DOTP, and (iii) allocating recycled content to the DOTP based at least in part on the tracing of recycled content along the chemical pathway.

In one aspect, the present technology concerns a process for producing dioctyl terephthalate (DOTP) having recycled content, where the process comprises the following steps: (a) producing a first syngas having physical recycled content from a waste plastic used to make the first syngas; (b) producing a second syngas having either no physical recycled content or less physical recycled content than the first syngas; (c) attributing recycled content credits from the waste plastic and/or from the first syngas to the second syngas; (d) feeding at least a portion of the second syngas to a DOTP production facility; and (e) producing recycled content dioctyl terephthalate (r-DOTP) in the DOTP production facility.

In one aspect, the present technology concerns a process for producing dioctyl terephthalate (DOTP) having recycled content, where the process comprises the following steps: (a) carbon reforming a first carbon-containing feed comprising waste plastic to thereby produce a first syngas having physical recycled content from the waste plastic; (b) booking recycled content credits attributable to the first syngas into a digital inventory; (c) gasifying a second carbon-containing feed comprising a solid hydrocarbon, a liquid hydrocarbon, and/or a gaseous hydrocarbon to thereby produce a second syngas having no physical recycled content attributable to waste plastic; (d) assigning recycled content credits from the digital inventory to the second syngas; (e) reacting at least a portion of the second syngas and propylene to form n-butyraldehyde; (f) converting at least a portion of the n-butyraldehyde to 2-ethylhexanol; and (g) reacting at least a portion of the 2-ethylhexanol with dimethyl terephthalate to thereby produce recycled content dioctyl terephthalate (r-DOTP).

### BRIEF DESCRIPTION OF THE DRAWINGS

FIG. 1 is a block flow diagram illustrating the main steps of a process and facility for making recycled content dioctyl terephthalate (r-DOTP), where the r-DOTP has credit-based recycled content from recycled content syngas (r-syngas);

FIG. 2 is a block flow diagram illustrating the main steps of a process and facility for making r-DOTP, where the r-DOTP has credit-based recycled content from r-syngas and physical recycled content from recycled content propylene (r-propylene);

FIG. 3 is a block flow diagram illustrating the main steps of a process and facility for making r-DOTP, where the r-DOTP has credit-based recycled content from r-syngas and physical recycled content from recycled content dimethyl terephthalate (r-DMT);

FIG. 4 is a block flow diagram illustrating the main steps of a process and facility for making r-DOTP, where the r-DOTP has credit-based recycled content from r-syngas and physical recycled content from r-propylene and r-DMT;

FIG. 5 is a block flow diagram illustrating the main steps of a process and facility for making r-DOTP that can have 100 percent total recycled content, where the r-DOTP has credit-based recycled content from r-syngas, r-propylene, and r-DMT;

FIG. 6 is a block flow diagram illustrating the main steps of a process and facility for making r-DOTP that can have 100 percent total recycled content, where the r-DOTP has credit-based recycled content from r-syngas and r-DMT and physical recycled content from r-propylene;

FIG. 7 is a block flow diagram illustrating the main steps of a process and facility for making r-DOTP that can have 100 percent total recycled content, where the r-DOTP has credit-based recycled content from r-syngas and r-propylene and physical recycled content from r-DMT; and

FIG. 8 is a block flow diagram illustrating the main steps of a process and facility for making r-DOTP that can have 100 percent total recycled content, where the r-DOTP has credit-based recycled content and/or physical recycled content from r-syngas, r-propylene, and r-DMT.

### DETAILED DESCRIPTION

We have discovered new methods and systems for producing dioctyl terephthalate (DOTP) having recycled content. More specifically, we have discovered a process and system for producing DOTP where recycled content from waste materials, such as waste plastic, are applied to DOTP in a manner that promotes the recycling of waste plastic and provides DOTP with up to 100 percent recycled content.

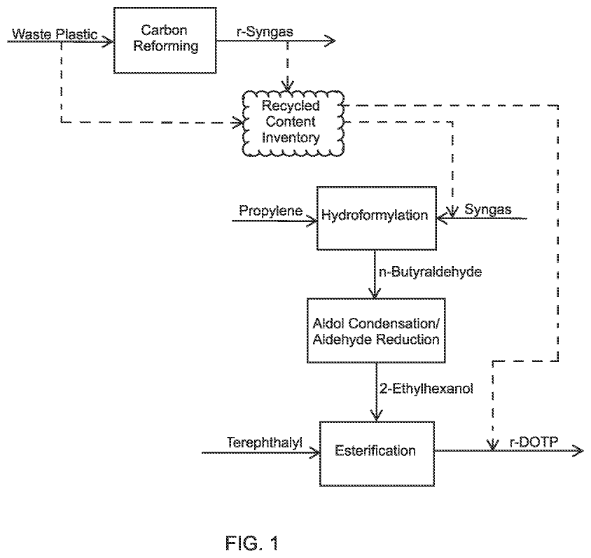

FIG. 1 depicts a process and system for producing recycled content dioctyl terephthalate (r-DOTP) in accordance with one embodiment of the present technology. As shown in FIG. 1 a waste material, such as waste plastic, can be subjected to carbon reforming to produce a recycled content syngas (r-syngas) having physical recycled content. In one embodiment, the feed to carbon reforming can comprise both a recycled content feed component (e.g., waste plastic) and a non-recycled content feed component (e.g., coal, a liquid hydrocarbon, and/or a gaseous hydrocarbon). In one embodiment, the carbon reforming is partial oxidation gasification that is fed with coal and waste plastic. In another embodiment, the carbon reforming is plasma gasification of a predominately waste plastic feed. In yet another embodiment, the carbon reforming is partial oxidation gasification fed with a non-recycled content liquid or gaseous hydrocarbon and a recycled content pyrolysis oil produced from the pyrolysis of waste plastic.

As described in further detail below, even though the waste plastic and r-syngas are not physically used to make the r-DOTP, recycled content from the r-syngas, the waste plastic, or both the r-syngas and waste plastic can ultimately be applied to DOTP to thereby provide r-DOTP.

In the embodiment of FIG. 1, DOTP is be made in a DOTP process/facility by a multi-step process that includes (a) hydroformylation of syngas and propylene to produce n-butyraldehyde, (b) aldol condensation and aldehyde reduction of the n-butyraldehyde to produce 2-ethylhexanol, and (c) esterification of the 2-ethylhexanol and a terephthalyl to produce the DOTP. The terephthalyl fed to esterification can be dimethyl terephthalate (DMT) or terephthalic acid (TPA).

In the process and system depicted in FIG. 1, the r-syngas produced by carbon reforming of waste plastic is not directly fed to hydroformylation. Rather, recycled content credits from the r-syngas product of carbon reforming are attributed to the syngas fed to hydroformylation. As such, the r-syngas from carbon reforming acts as a “source material” of recycled content credits and the syngas fed to hydroformylation acts as a “target material” to which the recycled content credits are attributed.

In one or more embodiment the source material has physical recycled content and the target material has less than 100 percent physical recycled content. For example, the source material can have at least 10, 25, 50, 75, 90, 99, or 100 percent physical recycled content and/or the target material can have less than 100, 99, 90, 75, 50, 25, 10, or 1 percent physical recycled content.

The ability to attribute recycled content credits from a source material to a target material removes the co-location requirement for the facility making the source material (with physical recycled content) and the facility making the DOTP. This allows a chemical recycling facility/site in one location to process waste material into one or more recycled content source materials and then apply recycled content credits from those source materials to one or more target materials being processed in existing commercial facilities located remotely from the chemical recycling facility/site. Further, the use of recycled content credits allows different entities to produce the source material and the r-DOTP. This allows efficient use of existing commercial assets to produce r-DOTP. In one or more embodiments, the source material is made at a facility/site that is at least 0.1, 0.5, 1, 5, 10, 50, 100, 500, or 1000 miles from the facility/site where the target material is used to make DOTP.

The attributing of recycled content credits from the source material (e.g., the r-syngas product from carbon reforming) to the target material (e.g., the syngas fed to hydroformylation) can be accomplished by transferring recycled content credits directly from the source material to the target material. Alternatively, as shown in FIG. 1, recycled content credits can be applied from the waste plastic, the r-syngas, or both the waste plastic and r-syngas to the DOTP via a recycled content inventory. The recycled content inventory can be a digital inventory or database used to record and track recycled content for various materials at various sites over various time periods.

When a recycled content inventory is used, recycled content credits from the source material having physical recycled content (e.g., the waste plastic and/or the r-syngas in FIG. 1) are booked into the recycled content inventory. The recycled content inventory can also contain recycled content from other sources and from other time periods. In one embodiment, recycled content credits in the recycled content inventory can only be assigned to target materials having the same or similar composition as the source materials. For example, as shown in FIG. 1, recycled content credits booked into the recycled content inventory from the r-syngas from carbon reforming can be assigned to the syngas fed to hydroformylation because the two syngas have the same or similar compositions. However, recycled content credits from r-syngas could not be assigned to the propylene fed to hydroformylation or the terephthalyl fed to esterification because the source and target materials would not be the same or similar.

Once recycled content credits have been attributed to the target material (e.g., the syngas fed to hydroformylation in FIG. 1), the amount of the credit-based recycled content allocated to the DOTP from the syngas is calculated by tracing the recycled content along the chemical pathway from the target material (e.g., the syngas fed to hydroformylation in FIG. 1) to the DOTP. The chemical pathway includes all chemical reactions and other processing steps (e.g., separations) between the target material and the DOTP.

In one or more embodiments, a conversion factor can be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of recycled content applied to the r-DOTP can be determined using one of variety of methods for quantifying, tracking, and allocating recycled content among various materials in various processes. One suitable method, known as “mass balance,” quantifies, tracks, and allocates recycled content based on the mass of the recycled content traced through the process. In certain embodiments the method of quantifying, tracking, and allocating recycled content is overseen by a certification entity that confirms the accuracy of the method and provides certification for the application of recycled content to the r-DOTP.

In one or more embodiments, the recycled content of the r-DOTP product can include physical recycled content and credit-based recycled content. As used herein, the term “total recycled content” refers to the cumulative amount of physical recycled content and credit-based recycled content from all sources.

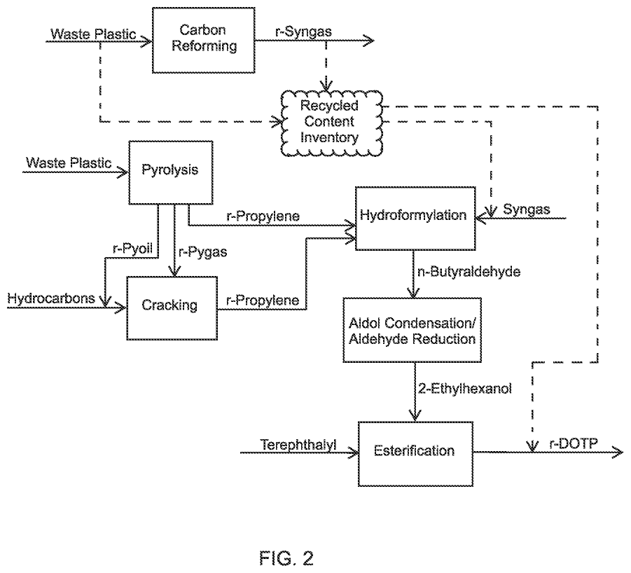

In the embodiment depicted in FIG. 1, the r-DOTP product can have a total recycled content of 5 to 60, 10 to 40, or 15 to 25 percent if, for example, (i) the syngas fed to hydroformylation has 100 percent credit-based recycled content and (ii) the propylene and terephthalyl fed to hydroformylation and esterification, respectively, have no recycled content. If none of the syngas, propylene, and terephthalyl have any physical recycled content, then all of the recycled content of the r-DOTP is credit-based recycled content. As discuss below with reference to FIGS. 2-8, the amount of recycled content applied to the DOTP product can be varied depending on the physical recycled content and/or credit-based recycled content of the syngas, propylene, and terephthalyl fed into the DOTP production process.

FIG. 2 illustrates a r-DOTP production process and system wherein credit-based recycled content is supplied via the syngas fed to hydroformylation and physical recycled content is supplied via recycled content propylene (r-propylene) fed to hydroformylation.

As depicted in FIG. 2, the r-propylene can be produced by pyrolyzing waste plastic to produce a pyrolysis effluent comprising, for example, r-propylene, r-pyoil, r-pygas, and/or heavier components (e.g., chars, tars, and waxes). In some cases, the r-propylene can exit the pyrolysis step as a component of the r-pygas. In other cases, the r-propylene can be separated from the r-pygas as part of the pyrolysis step, and the separated r-propylene can then be directly fed from pyrolysis to hydroformylation. In one or more embodiments, the r-pyoil can be co-fed with non-recycled content liquid or gaseous hydrocarbons to a cracking step to thereby produce a cracked effluent comprising r-propylene. In one or more embodiments, the r-pygas can be fed to a separation section of the cracking facility downstream of the cracking furnace for separation/recovery of r-propylene. The recovered r-propylene from the cracking facility can then be fed to hydroformylation. In the embodiment depicted in FIG. 2, the pyrolysis and cracking facilities are co-located with the DOTP production facility, while the carbon reforming facility is located remotely from the DOTP production facility.

In one or more embodiments, the r-DOTP has both credit-based recycled content (e.g., from the r-syngas of FIG. 2) and physical recycled content (e.g., from the r-propylene of FIG. 2). For example, the r-DOTP can have at least 10, 20, 30, 40, 50 percent physical recycled content and at least 10, 20, 30, 40, or 50 percent credit-based recycled content.

As discussed above, the amount of credit-based recycled content allocated from the target material (e.g., syngas in FIG. 2) to the r-DOTP is determined by tracing the credit-based recycled content along the chemical pathway from the target material to the DOTP. In a similar manner, the amount of physical recycled content allocated from the feed material having physical recycled content (e.g., r-propylene in FIG. 2) to the r-DOTP can be determined by tracing the physical recycled content along the chemical pathway from feed material to the DOTP. The chemical pathway includes all chemical reactions and other processing steps (e.g., separations) between the feed material having physical recycled content and the DOTP. In one or more embodiments, a conversion factor can be associate with each step along the chemical pathway of the physical recycled content. The conversion factors account for the amount of the physical recycled content diverted or lost at each step along the chemical pathway. For example, the conversion factors can account for the conversion, yield, and/or selectivity of the chemical reactions along the chemical pathway.

In the embodiment depicted in FIG. 2, the r-DOTP product can have a total recycled content of 30 to 95, 50 to 80, or 60 to 70 if, for example, (i) the syngas fed to hydroformylation has 100 percent credit-based recycled content, (ii) the propylene fed to hydroformylation has 100 percent physical recycled content, and (ii) the terephthalyl fed to esterification has no recycled content. In such a scenario, the r-DOTP can have both physical recycled content and credit-based recycle, including, for example, (i) 5 to 60, 10 to 40, or 15 to 25 percent credit-based recycled content from the r-syngas and (ii) 10 to 80, 25, to 65, or 40 to 50 percent physical recycled content from the r-propylene.

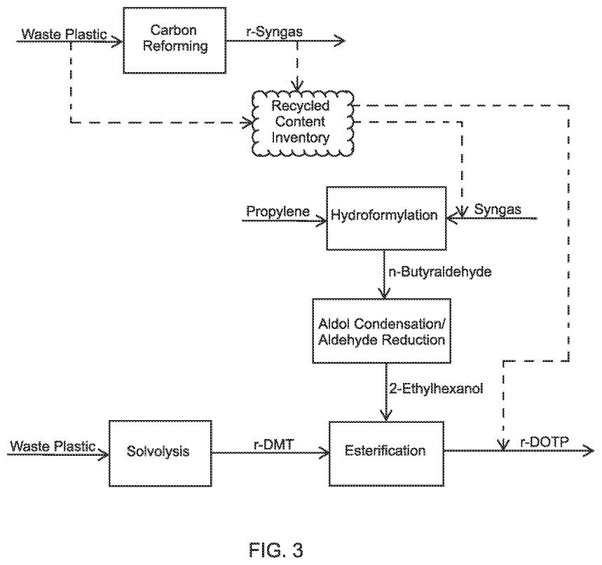

FIG. 3 illustrates a r-DOTP production process and system wherein credit-based recycled content is supplied via the syngas fed to hydroformylation and physical recycled content is supplied via recycled content dimethyl terephthalate (r-DMT) fed to esterification.

As depicted in FIG. 3, the r-DMT can be produced by solvolysis (e.g., methanolysis, alcoholysis, and/or hydrolysis) of waste plastic to produce the r-DMT fed to esterification. In one embodiment, the solvolysis step is methanolysis of polyethylene terephthalate (PET) waste plastic. The recovered r-DMT from the solvolysis facility can then be fed to esterification. In the embodiment depicted in FIG. 3, the solvolysis facility is co-located with the DOTP production facility, while the carbon reforming facility is located remotely from the DOTP production facility.

In the embodiment depicted in FIG. 3, the r-DOTP product can have a total recycled content of 25 to 90, 40 to 75, or 50 to 60 if, for example, (i) the syngas fed to hydroformylation has 100 percent credit-based recycled content, (ii) the propylene fed to hydroformylation has no recycled content, and (iii) the r-DMT fed to esterification has 100 percent physical recycled content. In such a scenario, the r-DOTP can have both physical recycled content and credit-based recycled content, including, for example, (i) 5 to 60, 10 to 40, or 15 to 25 percent credit-based recycled content from the r-syngas and (ii) 10 to 70, 20 to 55, or 30 to 40 percent physical recycled content from the r-DMT.

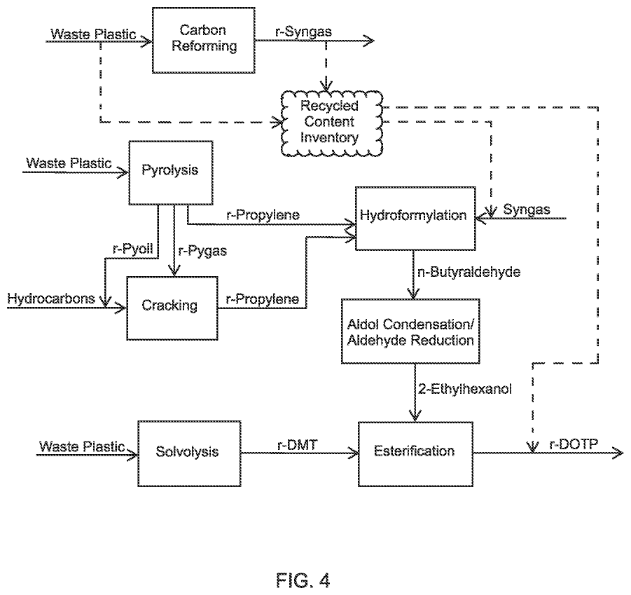

FIG. 4 illustrates a r-DOTP production process and system wherein credit-based recycled content is supplied via the syngas fed to hydroformylation and physical recycled content is supplied via r-propylene fed to hydroformylation and r-DMT fed to esterification.

In the embodiment depicted in FIG. 4, the r-propylene production facility and r-DMT production facility can be co-located with the r-DOTP production facility, while the r-syngas production facility can be located remotely from the r-DOTP production facility.

In the embodiment depicted in FIG. 4, the r-DOTP product can have a total recycled content of 100 percent if, for example, (i) the syngas fed to hydroformylation has 100 percent credit-based recycled content, (ii) the r-propylene fed to hydroformylation has 100 percent physical recycled content, and (iii) the r-DMT fed to esterfication has 100 percent physical recycled content. In such a scenario, the r-DOTP can have both physical recycled content and credit-based recycled content, including, for example, (i) 5 to 60, 10 to 40, or 15 to 25 percent credit-based recycled content from the r-syngas, (ii) 10 to 80, 25 to 65, or 40 to 50 percent physical recycled content from the r-propylene, and (iii) 10 to 70, 20 to 55, or 30 to 40 percent physical recycled content from the r-DMT.

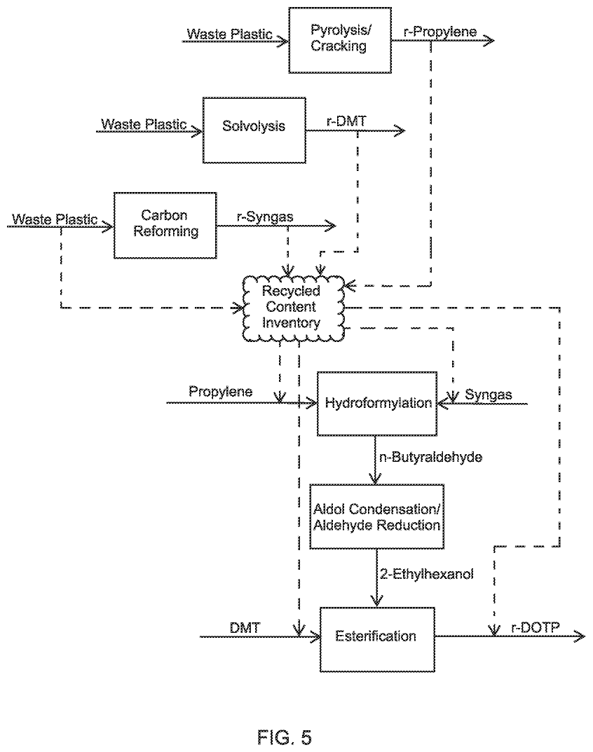

FIG. 5 illustrates a r-DOTP production process and system wherein credit-based recycled content is supplied via the syngas fed to hydroformylation, the propylene to hydroformylation, and the DMT fed to esterification. In this configuration, the r-syngas from carbon reforming, the r-propylene from pyrolysis/cracking, and the r-DMT from solvolysis and are all “source materials” having physical recycled content, while the syngas fed to hydroformylation, the propylene fed to hydroformylation, and the DMT fed to esterification are all “target materials” to which recycled content credits are attribute. The recycled content credits attributed to the syngas, the propylene, and the DMT target materials are from the r-syngas, r-propylene, and r-DMT source materials, respectively.

In the embodiment depicted in FIG. 5, the r-syngas production facility, the r-propylene production facility, and the r-DMT production facility can all be located remotely from the r-DOTP production facility.

In the embodiment depicted in FIG. 5, the r-DOTP product can have a total recycled content of 100 percent if, for example, (i) the syngas fed to hydroformylation has 100 percent credit-based recycled content, (ii) the propylene fed to hydroformylation has 100 percent credit-based recycled content, and (iii) the DMT fed to esterification has 100 percent credit-based recycled content. In such a scenario, the r-DOTP only has credit-based recycled content, including, for example, (i) 5 to 60, 10 to 40, or 15 to 25 percent credit-based recycled content from the r-syngas, (ii) 10 to 80, 25, to 65, or 40 to 50 percent credit-based recycled content from the r-propylene, and (iii) 10 to 70, 20 to 55, or 30 to 40 percent credit-based recycled content from the r-DMT.

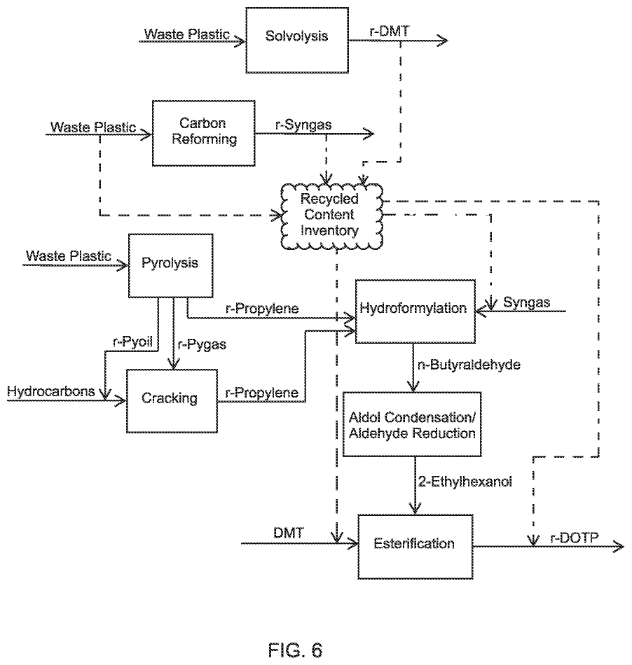

FIG. 6 illustrates a r-DOTP production process and system wherein credit-based recycled content is supplied via the syngas fed to hydroformylation and the DMT fed to esterification and physical recycled content is supplied via r-propylene fed to hydroformylation.

In the embodiment depicted in FIG. 6, the r-propylene production facility can be co-located with the r-DOTP production facility, while the r-syngas production facility and the r-DMT production facility can be located remotely from the r-DOTP production facility.

In the embodiment depicted in FIG. 6, the r-DOTP product can have a total recycled content of 100 percent if, for example, (i) the syngas fed to hydroformylation has 100 percent credit-based recycled content, (ii) the r-propylene fed to hydroformylation has 100 percent physical recycled content, and (iii) the r-DMT fed to esterification has 100 percent credit-based recycled content. In such a scenario, the r-DOTP can have both physical recycled content and credit-based recycled content, including, for example, (i) 5 to 60, 10 to 40, or 15 to 25 percent credit-based recycled content from the r-syngas, (ii) 10 to 80, 25 to 65, or 40 to 50 percent physical recycled content from the r-propylene, and (iii) 10 to 70, 20 to 55, or 30 to 40 percent credit-based recycled content from the r-DMT.

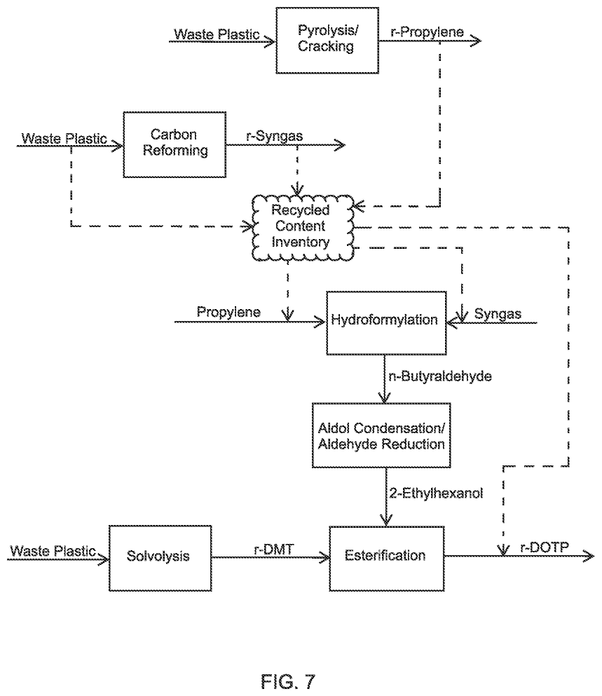

FIG. 7 illustrates a r-DOTP production process and system wherein credit-based recycled content is supplied via the syngas and propylene fed to hydroformylation and physical recycled content is supplied via the r-DMT fed to esterification.

In the embodiment depicted in FIG. 7, the r-DMT production facility can be co-located with the r-DOTP production facility, while the r-syngas production facility and the r-propylene production facility can be located remotely from the r-DOTP production facility.

In the embodiment depicted in FIG. 7, the r-DOTP product can have a total recycled content of 100 percent if, for example, (i) the syngas fed to hydroformylation has 100 percent credit-based recycled content, (ii) the propylene fed to hydroformylation has 100 percent credit-based recycled content, and (iii) the r-DMT fed to esterification has 100 percent physical recycled content. In such a scenario, the r-DOTP can have both physical recycled content and credit-based recycled content, including, for example, (i) 5 to 60, 10 to 40, or 15 to 25 percent credit-based recycled content from the r-syngas, (ii) 10 to 80, 25 to 65, or 40 to 50 percent credit-based recycled content from the r-propylene, and (iii) 10 to 70, 20 to 55, or 30 to 40 percent physical recycled content from the r-DMT.

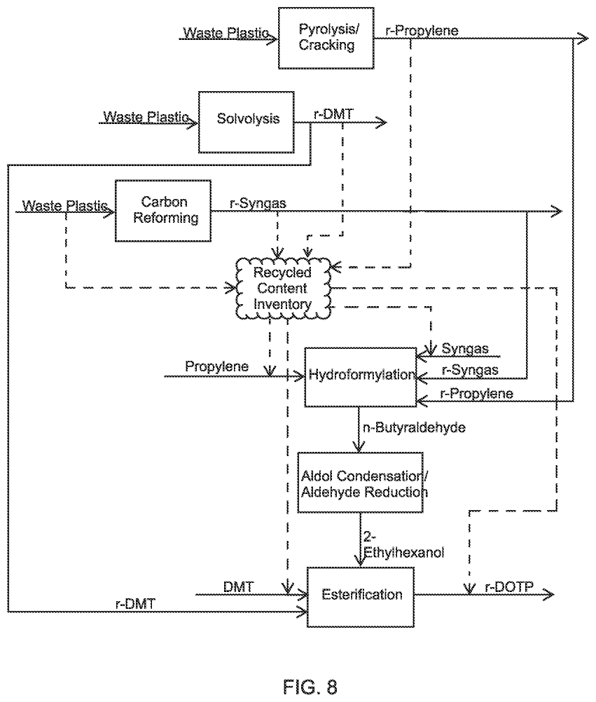

FIG. 8 illustrates a r-DOTP production process and system wherein credit-based recycled content and/or physical recycled content is supplied via the r-syngas/syngas to hydroformylation, the r-propylene/propylene to hydroformylation, and the r-DMT/DMT to esterification. FIG. 8 shows that the chemical reactions used to make r-DOTP can each be fed with the same type of stream, with one having physical recycled content and the other having credit-based recycled content. For example, the hydroformylation step can be fed with r-syngas having physical recycled content and with syngas having only credit-based recycled content, the hydroformylation step can be fed with r-propylene having physical recycled content and propylene having only credit-based recycled content, and the esterification step can be fed with r-DMT having physical recycled content and DMT having only credit-based recycled content.

In the embodiment depicted in FIG. 8, one r-syngas production facility, one r-propylene production facility, and one r-DMT production facility can be co-located with the r-DOTP production facility, while another r-syngas production facility, another r-propylene production facility, and another r-DMT production facility can be located remotely from the r-DOTP production facility.

In the embodiment depicted in FIG. 7, the r-DOTP can have a total recycled content of 100 percent if, for example, (i) the r-syngas and/or syngas fed to hydroformylation has 100 percent credit-based and/or recycled content, (ii) the r-propylene and/or propylene fed to hydroformylation has 100 percent credit-based and or physical recycled content, and (iii) the r-DMT and/or DMT fed to esterification has 100 percent credit-based and/or physical recycled content. In such a scenario, the r-DOTP can have both physical recycled content and credit-based recycled content, including (i) 5 to 60, 10 to 40, or 15 to 25 percent credit-based and/or physical recycled content from the r-syngas, (ii) 10 to 80, 25 to 65, or 40 to 50 percent credit-based and/or physical recycled content from the r-propylene, and (iii) 10 to 70, 20 to 55, or 30 to 40 percent credit-based and/or physical recycled content from the r-DMT.

### Claim Supporting Description—First Embodiment

In a first embodiment of the present technology there is provided a process for producing dioctyl terephthalate (DOTP) having recycled content, where the process comprises the following steps: (a) reacting a propylene with a syngas to thereby provide an n-butyraldehyde; (b) converting at least a portion of the n-butyraldehyde into a 2-ethylhexanol; (c) reacting at least a portion of the 2-ethylhexanol with a terephthalyl to thereby provide a DOTP; and (d) applying recycled content to at least a portion of the DOTP to thereby provide a recycled content DOTP (r-DOTP). The applying of step (d) includes (i) attributing recycled content from at least one source material having physical recycled content to at least one target material via a recycled content credit, (ii) tracing recycled content along at least one chemical pathway from the at least one target material to the DOTP, and (iii) allocating recycled content to the DOTP based at least in part on the tracing of recycled content along the chemical pathway.

The first embodiment described in the preceding paragraph can also include one or more of the additional aspects listed in the following paragraphs. The each of the following additional aspects of the first embodiment can be standalone features or can be combined with one or more of the other additional aspects to the extent consistent.

In an additional aspect of the first embodiment, the applying of step (d) uses mass balance.

In an additional aspect of the first embodiment, the process further comprises obtaining certification from a certification entity for the applying of step (d).

In an additional aspect of the first embodiment, the recycled content of the source material is from waste plastic.

In an additional aspect of the first embodiment, the source material and the target material comprise the same type of material.

In an additional aspect of the first embodiment, at least one of the following criterial is met (i) the source material and the target material both comprise syngas, (ii) the source material and the target material both comprise propylene, and/or (iii) the source material and the target material both comprise dimethyl terephthalate.

In an additional aspect of the first embodiment, the source material and the target material have substantially the same physical composition.

In an additional aspect of the first embodiment, at least 50, 75, 90, 95, 99, or 100 weight percent of the source material is identical to the target material.

In an additional aspect of the first embodiment, the attributing includes (i) booking recycled content credits attributable to the at least one source material into a digital inventory and (ii) assigning recycled content credits from the digital inventory to the target material.

In an additional aspect of the first embodiment, the tracing includes determining one or more conversion factors for one or more chemical reactions along the chemical pathway.

In an additional aspect of the first embodiment, the conversion factors account for the conversion, yield, and selectivity of the chemical reactions in the chemical pathway.

In an additional aspect of the first embodiment, the attributing includes assigning credit-based recycled content from a digital inventory to the target material and the conversion factors determine how much of the credit-based recycled content applied to the target material is allocated to the DOTP.

In an additional aspect of the first embodiment, the r-DOTP has a total recycled content of at least 10, 20, 30, 40, 50, 75, 90, 95, or 100 percent.

In an additional aspect of the first embodiment, the r-DOTP has both physical recycled content and credit-based recycled content.

In an additional aspect of the first embodiment, the r-DOTP has at least 10, 20, 30, 40, 50 percent physical recycled content and at least 10, 20, 30, 40, or 50 percent credit-based recycled content.

In an additional aspect of the first embodiment, the source material comprises a recycled content syngas (r-syngas) having physical recycled content and the target material comprises the syngas of step (a).

In an additional aspect of the first embodiment, the r-DOTP has 5 to 60, 10 to 40, or 15 to 25 percent credit-based recycled content from the r-syngas.

In an additional aspect of the first embodiment, the source material comprises a recycled content propylene (r-propylene) having physical recycled content and the target material comprises the propylene of step (a).

In an additional aspect of the first embodiment, the r-DOTP has 10 to 80, 25 to 65, or 40 to 50 percent credit-based recycled content from the r-propylene.

In an additional aspect of the first embodiment, the source material comprises a recycled content terephthalyl (r-terephthalyl) having physical recycled content and the target material comprises the terephthalyl of step (c).

In an additional aspect of the first embodiment, the r-DOTP has 10 to 70, 20 to 55, or 30 to 40 percent credit-based recycled content from the r-terephthalyl.

In an additional aspect of the first embodiment, the terephthalyl is at least one of dimethyl terephthalate (DMT) and/or terephthalic acid (TPA)

In an additional aspect of the first embodiment, the source material has physical recycled content and the target material has less than 100 percent physical recycled content.

In an additional aspect of the first embodiment, the source material has at least 10, 25, 50, 75, 90, or 99 percent physical recycled content.

In an additional aspect of the first embodiment, the target material has less than 99, 90, 75, 50, 25, 10, or 1 percent physical recycled content.

In an additional aspect of the first embodiment, the source material has 100 percent physical recycled content and the target material has no physical recycled content.

In an additional aspect of the first embodiment, none of the propylene, the syngas, and the terephthalyl have physical recycled content.

In an additional aspect of the first embodiment, at least one of the propylene, the syngas, and the terephthalyl has physical recycled content.

In an additional aspect of the first embodiment, at least two of the propylene, the syngas, and the terephthalyl have physical recycled content.

In an additional aspect of the first embodiment, all three of the propylene, the syngas, and the terephthalyl have physical recycled content.

In an additional aspect of the first embodiment, the applying further comprises applying physical recycled content to at least a portion of the DOTP so that the r-DOTP has both physical recycled content and credit-based recycled content.

In an additional aspect of the first embodiment, the physical recycled content applied to the DOTP is from at least one of the propylene, the syngas, and the terephthalyl.

In an additional aspect of the first embodiment, the target material comprises the syngas and at least a portion of the credit-based recycled content allocated to the DOTP is traced through a first chemical pathway from the syngas to the DOTP.

In an additional aspect of the first embodiment, at least a portion of the physical recycled content applied to the DOTP is from the propylene and is traced through a second chemical pathway from the propylene to the DOTP.

In an additional aspect of the first embodiment, the source material comprises at least one of (i) a waste plastic, (ii) a recycled content syngas (r-syngas) having physical recycled content from waste plastic, (iii) a recycled content propylene (r-propylene) having physical recycled content from waste plastic, and/or iv) a recycled content terephthalyl (r-terephthalyl) having physical recycled content from waste plastic.

In an additional aspect of the first embodiment, the target material comprises at least one of (i) the syngas, (ii) the propylene, and/or (iii) the terephthalyl.

In an additional aspect of the first embodiment, the r-DOTP has credit-based recycled content from the r-syngas.

In an additional aspect of the first embodiment, the r-DOTP has physical recycled content from at least one of the r-propylene and/or the r-terephthalyl.

In an additional aspect of the first embodiment, the source material comprises the r-syngas.

In an additional aspect of the first embodiment, the applying includes attributing credit-based recycled content from the r-syngas to the syngas via a digital inventory.

In an additional aspect of the first embodiment, the chemical pathway includes a hydroformylation reaction fed with the syngas and the propylene, an aldol condensation reaction fed with the n-butyraldehyde, an aldehyde reduction reaction fed with a product of the aldol condensation, and an esterification reaction fed with the 2-ethylhexanol and the terephthalyl.

In an additional aspect of the first embodiment, the process further comprises producing the r-syngas by carbon reforming a feed comprising a recycled content feed component.

In an additional aspect of the first embodiment, the recycled content feed component comprises waste plastic and/or a material obtained from waste plastic.

In an additional aspect of the first embodiment, the feed to the carbon reforming further comprises a non-recycled feed component.

In an additional aspect of the first embodiment, the non-recycled feed component comprises at least one of coal, a liquid hydrocarbon, and a gaseous hydrocarbon.

In an additional aspect of the first embodiment, the carbon reforming comprises partial oxidation gasification.

In an additional aspect of the first embodiment, at least a portion of the r-syngas is produced at a first site and the DOTP is produced at a second site.

In an additional aspect of the first embodiment, the first and second sites are space from one another by at least 0.1, 0.5, 1, 5, 10, 50, 100, 500, or 1000 miles.

In an additional aspect of the first embodiment, at least a portion of the r-syngas is produced by a different entity than the entity producing the DOTP.

In an additional aspect of the first embodiment, the source material comprises the r-propylene.

In an additional aspect of the first embodiment, the applying includes attributing credit-based recycled content from the r-propylene to the propylene via a digital inventory.

In an additional aspect of the first embodiment, the chemical pathway includes a hydroformylation reaction fed with the syngas and the propylene, an aldol condensation reaction fed with the n-butyraldehyde, an aldehyde reduction reaction fed with a product of the aldol condensation, and an esterification reaction fed with the 2-ethylhexanol and the terephthalyl.

In an additional aspect of the first embodiment, the process further comprises producing the r-propylene by pyrolysis and/or cracking of a feed comprising a recycled content feed component.

In an additional aspect of the first embodiment, the recycled content feed component comprises waste plastic and/or a material obtained from waste plastic.

In an additional aspect of the first embodiment, the feed to the pyrolysis and/or cracking further comprises a non-recycled feed component.

In an additional aspect of the first embodiment, the non-recycled feed component comprises at least one of a liquid hydrocarbon and a gaseous hydrocarbon.

In an additional aspect of the first embodiment, at least a portion of the r-propylene is produced via pyrolysis of waste plastic.

In an additional aspect of the first embodiment, the pyrolysis produces a pyrolysis gas comprising the r-propylene.

In an additional aspect of the first embodiment, the producing of the r-propylene includes pyrolysis of waste plastic to produce a pyrolysis effluent having recycled content and then cracking at least a portion of the pyrolysis effluent to thereby provide at least a portion of the r-propylene.

In an additional aspect of the first embodiment, at least a portion of the r-propylene is produced at a first site and the DOTP is produced at a second site, In an additional aspect of the first embodiment, the first and second sites are space from one another by at least 0.1, 0.5, 1, 5, 10, 50, 100, 500, or 1000 miles.

In an additional aspect of the first embodiment, at least a portion of the r-propylene is produced by a different entity than the entity producing the DOTP.

In an additional aspect of the first embodiment, the source material comprises the r-terephthalyl.

In an additional aspect of the first embodiment, the applying includes attributing credit-based recycled content from the r-terephthalyl to the terephthalyl via a digital inventory.

In an additional aspect of the first embodiment, the chemical pathway includes an esterification reaction fed with the 2-ethylhexanol and the terephthalyl.

In an additional aspect of the first embodiment, the process further comprises producing the r-terephthalyl by solvolysis of a feed comprising a recycled content feed component.

In an additional aspect of the first embodiment, the r-terephthalyl is recycled content dimethyl terephthalate (r-DMT).

In an additional aspect of the first embodiment, the solvolysis is methanolysis.

In an additional aspect of the first embodiment, the recycled content feed component comprises waste plastic and/or a material obtained from waste plastic.

In an additional aspect of the first embodiment, at least a portion of the r-terephthalyl is produced at a first site and the DOTP is produced at a second site, In an additional aspect of the first embodiment, the first and second sites are space from one another by at least 0.1, 0.5, 1, 5, 10, 50, 100, 500, or 1000 miles.

In an additional aspect of the first embodiment, at least a portion of the r-terephthalyl is produced by a different entity than the entity producing the DOTP.

In an additional aspect of the first embodiment, the r-DOTP has a total recycled content of 100 percent.

In an additional aspect of the first embodiment, a first portion of the total recycled content is attributable to a recycled content syngas (r-syngas) having physical recycled content, a second portion of the total recycled content is attributable to a recycled content propylene (r-propylene) having physical recycled content, and a third portion of the total recycled content is attributable to a recycled content terephthalyl (r-terephthalyl) having physical recycled content.

In an additional aspect of the first embodiment, the first portion of the total recycled content is 10 to 40 percent, the second portion of the total recycled content is 25 to 65 percent, and the third portion of the total recycled content is 20 to 55 percent.

In an additional aspect of the first embodiment, at least one of the following criteria is met (i) the r-syngas is the at least one source material and the target material comprises the syngas of step (a), (ii) the r-propylene is the at least on source material and the target material comprises the propylene of step (a), and (iii) the r-terephthalyl is the at least one source material and the target material comprises the terephthalyl of step (c).

In an additional aspect of the first embodiment, at least one of the following criteria is met (i) the syngas of step (a) is the r-syngas having physical recycled content, (ii) the propylene of step (a) is the r-propylene having physical recycled content, and (iii) the terephthalyl of step (c) is the r-terephthalyl having physical recycled content.

### Claim Supporting Description—Second Embodiment

In a second embodiment of the present technology there is provided a process for producing dioctyl terephthalate (DOTP) having recycled content, where the process comprises the following steps: (a) producing a first syngas having physical recycled content from a waste plastic used to make the first syngas; (b) producing a second syngas having either no physical recycled content or less physical recycled content than the first syngas; (c) attributing recycled content credits from the waste plastic and/or from the first syngas to the second syngas; (d) feeding at least a portion of the second syngas to a DOTP production facility; and (e) producing recycled content dioctyl terephthalate (r-DOTP) in the DOTP production facility.

The second embodiment described in the preceding paragraph can also include one or more of the additional aspects listed in the in the following paragraphs. The each of the following additional aspects of the second embodiment can be standalone features or can be combined with one or more of the other additional aspects to the extent consistent.

In an additional aspect of the second embodiment, at least a portion of the recycled content credits are attributable to the first syngas.

In an additional aspect of the second embodiment, the second syngas has no physical recycled content.

In an additional aspect of the second embodiment, the r-DOTP has 5 to 60, 10 to 40, or 15 to 25 percent credit-based recycled content.

In an additional aspect of the second embodiment, the process further comprises prior to step (a), receiving waste plastic into a first facility where the first syngas is produced and booking recycled content credits attributable to the waste plastic and/or the first syngas into a digital inventory.

In an additional aspect of the second embodiment, steps (a) and (b) are carried out by the same entity at different sites space from one another by at least 0.1, 0.5, 1, 5, 10, 50, 100, 500, or 1000 miles.

In an additional aspect of the second embodiment, steps (a) and (b) are carried out by different entities at different sites space from one another by at least 0.1, 0.5, 1, 5, 10, 50, 100, 500, 1000 miles.

In an additional aspect of the second embodiment, the producing of step (e) includes (i) reacting at least a portion of the second syngas and propylene to form n-butyraldehyde, (ii) converting at least a portion of the n-butyraldehyde to 2-ethylhexanol, and (iii) reacting the 2-ethylhexanol with dimethyl terephthalate or terephthalic acid to produce the r-DOTP.

In an additional aspect of the second embodiment, the producing of step (a) includes carbon reforming at least a portion of the waste plastic to thereby provide at least a portion of the first syngas.

In an additional aspect of the second embodiment, the carbon reforming comprises gasification.

In an additional aspect of the second embodiment, a feed to the gasification contains at least a portion of the waste plastic.

In an additional aspect of the second embodiment, the feed to gasification also comprises a carbon source selected from the group consisting of coal, a liquid hydrocarbon, a gaseous hydrocarbon and combinations thereof.

In an additional aspect of the second embodiment, the process further comprises tracing recycled content along a chemical pathway from the second syngas to the r-DOTP.

In an additional aspect of the second embodiment, the process further comprises allocating credit-based recycled content from the second syngas to the r-DOTP based at least in part on the tracing of recycled content along the chemical pathway.

In an additional aspect of the second embodiment, the tracing and allocating is carried out using mass balance.

In an additional aspect of the second embodiment, the tracing includes determining one or more conversion factors for one or more chemical reactions along the chemical pathway.

In an additional aspect of the second embodiment, the conversion factors account for the conversion, yield, and selectivity of the chemical reactions in the chemical pathway.

In an additional aspect of the second embodiment, the conversion factors determine how much of the credit-based recycled content assigned to the second syngas is allocated to the DOTP.

In an additional aspect of the second embodiment, the assigning of step (c) comprises booking recycled content credits attributable to the first syngas into a digital inventory and assigning recycled content credits from the digital inventory to the second syngas.

In an additional aspect of the second embodiment, the process further comprises subjecting a first carbon-containing feed comprising waste plastic to carbon reforming in a first conversion facility to thereby produce the first syngas having physical recycled content.

In an additional aspect of the second embodiment, the process further comprises subjecting a second carbon-containing feed comprising one or more hydrocarbons to partial oxidation gasification in a second conversion facility to thereby produce the second syngas.

In an additional aspect of the second embodiment, the carbon reforming is partial oxidation gasification.

In an additional aspect of the second embodiment, the first and second conversion facilities are spaced from on another by at least 0.1, 0.5, 1, 5, 10, 50, 100, 500, or 1000 miles.

In an additional aspect of the second embodiment, the process further comprises obtaining recycled content propylene (r-propylene) having physical recycled content and reacting at least a portion of the r-propylene with at least a portion of the second syngas to produce n-butyraldehyde.

In an additional aspect of the second embodiment, the obtaining includes pyrolyzing and/or cracking waste plastic thereby provide at least a portion of the r-propylene.

In an additional aspect of the second embodiment, the obtaining includes receiving at least a portion of the r-propylene from an entity or facility other than the entity or facility producing the r-DOTP.

In an additional aspect of the second embodiment, the process further comprises obtaining recycled content terephthalyl (r-terephthalyl) having physical recycled content and reacting at least a portion of the r-terephthalyl with 2-ethylhexanol to produce the r-DOTP.

In an additional aspect of the second embodiment, the r-terephthalyl comprises r-DMT and the obtaining includes solvolysis of waste plastic to thereby provide at least a portion of the r-DMT.

In an additional aspect of the second embodiment, the solvolysis comprises methanolysis of waste polyethylene terephthalate.

In an additional aspect of the second embodiment, the obtaining includes receiving at least a portion of the r-terephthalyl from an entity or facility other than the entity or facility producing the r-DOTP.

In an additional aspect of the second embodiment, the process further comprises attributing recycled content from a first propylene having physical recycled content to a second propylene not having physical recycled content and reacting at least a portion of the second propylene with the at least a portion of second syngas to produce n-butyraldehyde.

In an additional aspect of the second embodiment, the process further comprises pyrolyzing and/or cracking waste plastic to thereby provide at least a portion of the first propylene.

In an additional aspect of the second embodiment, the process further comprises receiving at least a portion of the first propylene from an entity or facility other than the entity or facility producing the r-DOTP.

In an additional aspect of the second embodiment, the process further comprises attributing recycled content from a first dimethyl terephthalate (DMT) having physical recycled content to a second DMT not having recycled content and reacting at least a portion of the second DMT with 2-ethylhexanol to thereby produce the r-DOTP.

In an additional aspect of the second embodiment, the process further comprises conducting solvolysis of waste plastic to thereby provide at least a portion of the first DMT.

In an additional aspect of the second embodiment, the solvolysis comprises methanolysis of waste polyethylene terephthalate.

In an additional aspect of the second embodiment, the process further comprises receiving at least a portion of the first DMT from an entity or facility other than the entity or facility producing the r-DOTP.

### Claim Supporting Description—Third Embodiment

In a third embodiment of the present technology there is provided a process for producing dioctyl terephthalate (DOTP) having recycled content, where the process comprises the following steps: (a) carbon reforming a first carbon-containing feed comprising waste plastic to thereby produce a first syngas having physical recycled content from the waste plastic; (b) booking recycled content credits attributable to the first syngas into a digital inventory; (c) gasifying a second carbon-containing feed comprising a solid hydrocarbon, a liquid hydrocarbon, and/or a gaseous hydrocarbon to thereby produce a second syngas having no physical recycled content attributable to waste plastic; (d) assigning recycled content credits from the digital inventory to the second syngas; (e) reacting at least a portion of the second syngas and propylene to form n-butyraldehyde; (f) converting at least a portion of the n-butyraldehyde to 2-ethylhexanol; and (g) reacting at least a portion of the 2-ethylhexanol with dimethyl terephthalate to thereby produce recycled content dioctyl terephthalate (r-DOTP).

The third embodiment described in the preceding paragraph can also include one or more of the additional aspects listed in the in the following paragraphs. The each of the following additional aspects of the third embodiment can be standalone features or can be combined with one or more of the other additional aspects to the extent consistent.

In an additional aspect of the third embodiment, the r-DOTP has a total recycled content of at least 10, 20, 30, 40, 50, 75, 90, 95, or 100 percent.

In an additional aspect of the third embodiment, the r-DOTP has 5 to 60, 10 to 40, or 15 to 25 percent credit-based recycled content from the first syngas.

In an additional aspect of the third embodiment, the r-DOTP has both physical recycled content and credit-based recycled content.

In an additional aspect of the third embodiment, the r-DOTP has at least 10, 20, 30, 40, or 50 percent physical recycled content and at least 10, 20, 30, 40, or 50 percent credit-based recycled content.

In an additional aspect of the third embodiment, steps (a) and (c) each comprise partial oxidation gasification.

In an additional aspect of the third embodiment, the first carbon-containing feed comprises coal and waste plastic.

In an additional aspect of the third embodiment, said second carbon-containing feed comprises a liquid hydrocarbon and/or a gaseous hydrocarbon.

In an additional aspect of the third embodiment, steps (a) and (c) are carried out in reactors that are spaced from one another by at least 0.1, 0.5, 1, 5, 10, 50, 100, 500, or 1000 miles.

In an additional aspect of the third embodiment, steps (a) and (b) are carried out by different entities at different sites spaced from one another by at least 0.1, 0.5, 1, 5, 10, 50, 100, 500, or 1000 miles.

In an additional aspect of the third embodiment, the process further comprises obtaining recycled content propylene (r-propylene) having physical recycled content and reacting at least a portion of the r-propylene with at least a portion of the second syngas to produce n-butyraldehyde.

In an additional aspect of the third embodiment, the obtaining includes pyrolyzing and/or cracking waste plastic thereby provide at least a portion of the r-propylene.

In an additional aspect of the third embodiment, the obtaining includes receiving at least a portion of the r-propylene from an entity or facility other than the entity or facility producing the r-DOTP.

In an additional aspect of the third embodiment, the process further comprises obtaining recycled content dimethyl terephthalate (r-DMT) having physical recycled content and reacting at least a portion of the r-DMT with 2-ethylhexanol to produce the r-DOTP.

In an additional aspect of the third embodiment, the obtaining includes solvolysis of waste plastic to thereby provide at least a portion of the r-DMT.

In an additional aspect of the third embodiment, the solvolysis comprises methanolysis of waste polyethylene terephthalate.

In an additional aspect of the third embodiment, the obtaining includes receiving at least a portion of the r-DMT from an entity or facility other than the entity or facility producing the r-DOTP.

In an additional aspect of the third embodiment, the process further comprises attributing recycled content from a first propylene having physical recycled content to a second propylene not having physical recycled content and reacting at least a portion of the second propylene with the at least a portion of second syngas to produce n-butyraldehyde.

In an additional aspect of the third embodiment, the process further comprises pyrolyzing and/or cracking waste plastic to thereby provide at least a portion of the first propylene.

In an additional aspect of the third embodiment, the process further comprises receiving at least a portion of the first propylene from an entity or facility other than the entity or facility producing the r-DOTP.

In an additional aspect of the third embodiment, the process further comprises attributing recycled content from a first dimethyl terephthalate (DMT) having physical recycled content to a second DMT not having recycled content and reacting at least a portion of the second DMT with 2-ethylhexanol to thereby produce the r-DOTP.

In an additional aspect of the third embodiment, the process further comprises conducting solvolysis of waste plastic to thereby provide at least a portion of the first DMT.

In an additional aspect of the third embodiment, the solvolysis comprises methanolysis of waste polyethylene terephthalate.

In an additional aspect of the third embodiment, the process further comprises receiving at least a portion of the first DMT from an entity or facility other than the entity or facility producing the r-DOTP.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the term “co-located” refers to the characteristic of at least two objects being situated on a common physical site, and/or within one mile of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycled content based on the mass of the recycled content in various materials.

As used herein, the term “partial oxidation (POX) gasification” or “POX gasification” refers to high temperature conversion of a carbon-containing feed into syngas, (carbon monoxide, hydrogen, and carbon dioxide), where the conversion is carried out in the presence of a less than stoichiometric amount of oxygen. The feed to POX gasification can include solids, liquids, and/or gases.

As used herein, the terms “physical recycled content” and “direct recycled content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “pyrolysis” refers to thermal decomposition of one or more organic materials at elevated temperatures in an inert (i.e., substantially oxygen free) atmosphere.

As used herein, the term “pyrolysis gas” refers to a composition obtained from pyrolysis that is gaseous at 25° C.

As used herein, the terms “pyrolysis oil” or “pyoil” refers to a composition obtained from pyrolysis that is liquid at 25° C. and 1 atm.

As used herein, the term “pyrolysis residue” refers to a composition obtained from pyrolysis that is not pyrolysis gas or pyrolysis oil and that comprises predominantly pyrolysis char and pyrolysis heavy waxes.

As used herein, the term “recycled content” refers to being or comprising a composition that is directly and/or indirectly derived from recycle material. Recycled content is used generically to refer to both physical recycled content and credit-based recycled content. Recycled content is also used as an adjective to describe material having physical recycled content and/or credit-based recycled content.

As used herein, the term “recycled content credit” refers to a non-physical measure of physical recycled content that can be directly or indirectly (i.e., via a digital inventory) attributed from a first material having physical recycled content to a second material having less than 100 percent physical recycled content.

As used herein, the term “solvolysis” or “ester solvolysis” refers to a reaction by which an ester-containing feed is chemically decomposed in the presence of a solvent to form a principal carboxyl product and/or a principal glycol product. Examples of solvolysis include, hydrolysis, alcoholysis, and ammonolysis.

As used herein, the term “terephthalyl” refers to a molecule including the following group:

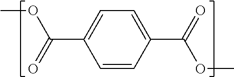

As used herein, the term “principal terephthalyl” refers to the main or key terephthalyl product being recovered from a solvolysis process.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing dioctyl terephthalate (DOTP) having recycled content, the process comprising:
(a) reacting a propylene with a syngas to thereby provide an n-butyraldehyde;
(b) converting at least a portion of the n-butyraldehyde into a 2-ethylhexanol;
(c) reacting at least a portion of the 2-ethylhexanol with a terephthalyl to thereby provide a DOTP; and
(d) applying recycled content to at least a portion of the DOTP to thereby provide a recycled content DOTP (r-DOTP),
wherein the applying of step (d) includes (i) attributing recycled content from at least one source material having physical recycled content to at least one target material via recycled content credits, (ii) tracing recycled content along at least one chemical pathway from the at least one target material to the DOTP, and (iii) allocating recycled content to the DOTP based at least in part on the tracing of recycled content along the chemical pathway.

2. The process of claim 1, wherein the applying of step (d) uses mass balance.

3. The process of claim 1, further comprising obtaining certification from a certification entity for the applying of step (d).

4. The process of claim 1, wherein the recycled content of the source material is from waste plastic.

5. The process of claim 1, wherein at least one of the following criterial is met (i) the source material and the target material both comprise syngas, (ii) the source material and the target material both comprise propylene, and/or (iii) the source material and the target material both comprise dimethyl terephthalate.

6. The process of claim 1, wherein the attributing includes (i) booking recycled content credits attributable to the at least one source material into a digital inventory and (ii) assigning recycled content credits from the digital inventory to the target material.

7. The process of claim 1, wherein the tracing includes determining one or more conversion factors for one or more chemical reactions along the chemical pathway,
wherein the attributing includes assigning credit-based recycled content from a digital inventory to the target material,
wherein the conversion factors determine how much of the credit-based recycled content applied to the target material is allocated to the DOTP.

8. The process of claim 1, wherein the r-DOTP has a total recycled content of at least 20 percent.

9. The process of claim 1, wherein the r-DOTP has at least 10 percent physical recycled content and at least 10 percent credit-based recycled content.

10. The process of claim 1, wherein at least one of the following criteria (i)-(iii) is met:
(i) the source material comprises a recycled content syngas (r-syngas) having physical recycled content, the target material comprises the syngas of step (a), and the r-DOTP has 5 to 60 percent credit-based recycled content from the r-syngas,
(ii) the source material comprises a recycled content propylene (r-propylene) having physical recycled content, the target material comprises the propylene of step (a), and the r-DOTP has 10 to 80 percent credit-based recycled content from the r-propylene, and/or
(iii) the source material comprises a recycled content terephthalyl (r-terephthalyl) having physical recycled content, the target material comprises the terephthalyl of step (c), and the r-DOTP has 10 to 70 percent credit-based recycled content from the r-terephthalyl.

11. The process of claim 1, wherein the applying further comprises applying physical recycled content to at least a portion of the DOTP so that the r-DOTP has both physical recycled content and credit-based recycled content, wherein the physical recycled content applied to the DOTP is from at least one of the propylene, the syngas, and the terephthalyl.

12. The process of claim 11, wherein the target material comprises the syngas and at least a portion of the credit-based recycled content allocated to the DOTP is traced through a first chemical pathway from the syngas to the DOTP, wherein at least a portion of the physical recycled content applied to the DOTP is from the propylene and is traced through a second chemical pathway from the propylene to the DOTP.

13. The process of claim 1, wherein the source material comprises at least one of (i) a waste plastic, (ii) a recycled content syngas (r-syngas) having physical recycled content from waste plastic, (iii) a recycled content propylene (r-propylene) having physical recycled content from waste plastic, and/or iv) a recycled content terephthalyl (r-terephthalyl) having physical recycled content from waste plastic, wherein the target material comprises at least one of (i) the syngas, (ii) the propylene, and/or (iii) the terephthalyl.

14. The process of claim 13, wherein the source material comprises the r-syngas, wherein the applying includes attributing credit-based recycled content from the r-syngas to the syngas via a digital inventory, wherein the chemical pathway includes a hydroformylation reaction fed with the syngas and the propylene, an aldol condensation reaction fed with the n-butyraldehyde, an aldehyde reduction reaction fed with a product of the aldol condensation, and an esterification reaction fed with the 2-ethylhexanol and the terephthalyl.

15. The process of claim 14, further comprising producing the r-syngas by carbon reforming a feed comprising a recycled content feed component, wherein the recycled content feed component comprises waste plastic and/or a material obtained from waste plastic.

16. The process of claim 1, wherein at least a portion of the r-syngas is produced at a first site and the DOTP is produced at a second site, wherein the first and second sites are space from one another by at least 0.1 miles.

17. The process of claim 1, wherein the r-DOTP has a total recycled content of 100 percent,
wherein a first portion of the total recycled content is attributable to a recycled content syngas (r-syngas) having physical recycled content,
wherein a second portion of the total recycled content is attributable to a recycled content propylene (r-propylene) having physical recycled content,
wherein a third portion of the total recycled content is attributable to a recycled content terephthalyl (r-terephthalyl) having physical recycled content,
wherein the first portion of the total recycled content is 10 to 40 percent, the second portion of the total recycled content is 25 to 65 percent, and the third portion of the total recycled content is 20 to 55 percent,
wherein at least one of the following criteria is met (i) the r-syngas is the at least one source material and the target material comprises the syngas of step (a), (ii) the r-propylene is the at least on source material and the target material comprises the propylene of step (a), and (iii) the r-terephthalyl is the at least one source material and the target material comprises the terephthalyl of step (c),
wherein at least one of the following criteria is met (i) the syngas of step (a) is the r-syngas having physical recycled content, (ii) the propylene of step (a) is the r-propylene having physical recycled content, and (iii) the terephthalyl of step (c) is the r-terephthalyl having physical recycled content.

18. A process for producing dioctyl terephthalate (DOTP) having recycled content, the process comprising:
(a) producing a first syngas having physical recycled content from a waste plastic used to make the first syngas;
(b) producing a second syngas having either no physical recycled content or less physical recycled content than the first syngas;
(c) attributing recycled content credits from the waste plastic and/or from the first syngas to the second syngas;
(d) feeding at least a portion of the second syngas to a DOTP production facility; and
(e) producing recycled content dioctyl terephthalate (r-DOTP) in the DOTP production facility.

19. The process of claim 1, wherein the r-DOTP has 5 to 60 percent credit-based recycled content attributable to the first syngas.

20. A process for producing dioctyl terephthalate (DOTP) having recycled content, the process comprising:
(a) carbon reforming a first carbon-containing feed comprising waste plastic to thereby produce a first syngas having physical recycled content from the waste plastic;
(b) booking recycled content credits attributable to the first syngas into a digital inventory;
(c) gasifying a second carbon-containing feed comprising a solid hydrocarbon, a liquid hydrocarbon, and/or a gaseous hydrocarbon to thereby produce a second syngas having no physical recycled content attributable to waste plastic;
(d) assigning recycled content credits from the digital inventory to the second syngas;
(e) reacting at least a portion of the second syngas and propylene to form n-butyraldehyde;
(f) converting at least a portion of the n-butyraldehyde to 2-ethylhexanol; and
(g) reacting at least a portion of the 2-ethylhexanol with dimethyl terephthalate to thereby produce recycled content dioctyl terephthalate (r-DOTP) having 10 to 40 percent credit-based recycled content from the first syngas.
